(19) United States
(12) Patent Application Publication
(10) Pub. No.: US 2014/0230818 A1
(43) Pub. Date: Aug. 21, 2014

(54) METHODS FOR PREDICTION OF VENTILATION TREATMENT INADEQUACY

(71) Applicant: COVIDIEN LP, Mansfield, MA (US)

(72) Inventors: Mehdi Jafari, Laguna Hills, CA (US); Peter Doyle, Vista, CA (US)

(73) Assignee: Covidien LP, Mansfield, MA (US)

(21) Appl. No.: 13/768,299

(22) Filed: Feb. 15, 2013

(51) Int. Cl. A61M 16/00; A61M 16/04; A61M 16/06; A61M 16/08

(52) U.S. Cl. CPC A61M 16/0051; A61M 16/0057; A61M 16/0875; A61M 16/04; A61M 16/06
USPC 128/204.23

(57) ABSTRACT

Ventilatory systems and methods are disclosed including a ventilator and at least one sensor in communication with the ventilator. The sensor is configured to monitor a chest wall movement of a patient during ventilation. The ventilator is configured to provide an alarm to a clinician upon detection by the sensor of a threshold change in the chest wall movement of the patient, for example, a threshold change between the monitored chest wall movement and a previously monitored chest wall movement of the patient. The sensor may be located on an exterior surface of the patient's chest and may be, for example, an accelerometer, an optical imaging system, an audio sensor, an electrical impedance sensor or an ultrasound sensor.

### TECHNICAL FIELD

The present disclosure relates generally to medical devices and to systems and methods for detecting inadequate ventilation treatment.

### BACKGROUND

A ventilator is a device that mechanically helps patients breathe by replacing some or all of the muscular effort required to inflate and deflate the lungs. In recent years, there has been an accelerated trend towards an integrated clinical environment. That is, medical devices are becoming increasingly integrated with communication, computing, and control technologies. As a result, modern ventilatory equipment has become increasingly complex, providing for detection and evaluation of a myriad of ventilatory parameters. However, due to the magnitude of available ventilatory data, many clinicians may not readily assess and evaluate the diverse ventilatory data to detect certain patient conditions and/or changes in patient conditions, such as inadequacy of ventilation treatment.

### SUMMARY

In accordance with aspects of the present disclosure, a ventilatory system is disclosed including a ventilator and a sensing system in communication with the ventilator. The sensing system is configured to monitor a chest wall movement of a patient during ventilation. The ventilator is configured to provide an alarm to a clinician upon detection by the sensor of a threshold change in the chest wall movement of the patient, for example, a threshold change between an indicator of the monitored chest wall movement and a previously monitored chest wall movement. The sensing system may be an integrated system consisting of multiple distributed sensing mechanisms or a sensor located on an exterior surface of the patient's chest and may be, for example, an accelerometer, an optical imaging system, an audio sensor, an electrical impedance sensor, an ultrasound sensor, or other similar sensors capable of determining the direction and magnitude of a patient's chest wall movement.

In accordance with other aspects of the present disclosure, a method of detecting inadequate ventilation of a patient is disclosed including monitoring a chest wall movement of the patient; determining if a threshold change in the chest wall movement of the patient has occurred; and triggering an alarm if the threshold change in the chest wall movement of the patient has occurred. The method may further include evaluating the characteristics (e.g., amplitude, frequency, direction, magnitude, symmetry, etc.) of the chest wall movement of the patient and comparing and trending parameters of such characteristics of the chest wall movement to corresponding parameters of previous chest wall movements of the patient; and triggering an alarm if the difference between the chest wall movement and the previous chest wall movement exceeds a pre-determined threshold.

In accordance with other aspects of the present disclosure, a non-transitory computer readable storage medium for storing computer-executable instructions for controlling a processor to execute a method of detecting inadequate ventilation of a patient is disclosed including computer-executable instructions for monitoring a chest wall movement of the patient; computer-executable instructions for determining if a threshold change in the chest wall movement of the patient has occurred; and computer-executable instructions for triggering an alarm if the threshold change in the chest wall movement of the patient has occurred.

The present disclosure provides new and unique advantages to ventilator treatment over prior ventilator systems. Monitoring a patient's chest movement through the use of external sensors allows a ventilator to make determinations on the adequacy of ventilator treatment based on an additional data parameter that was not previously available. This allows the ventilator to provide a more accurate assessment of a patient's condition for use by a clinician. In addition, monitoring patient chest movement by the ventilator removes the need for a clinician to make a continuous bed side observation of chest movement. This provides an added benefit in short staffing situations by reducing the amount of time a clinician must stay by the bed side to assess the patient. The use of chest movement monitoring also allows the ventilator to provide more targeted intelligent alarms to the clinician regarding ventilation inadequacy and to provide targeted recommendations or likely causes of the alarm situation to the clinician. Providing the clinician with targeted alarms allows the clinician to spend less time at the patient's bed side assessing the problem and significantly increases the efficiency with which the cause of the alarm can be assessed and addressed.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described herein below with reference to the accompanying drawings, wherein:

FIG. 1 is a diagram illustrating an embodiment of a ventilator connected to a patient;

FIG. 2 is a block diagram illustrating an embodiment of a ventilatory system, in accordance with an aspect of the present disclosure;

FIG. 3 is a perspective view of the torso of a patient illustrating sensors positioned on the patient's chest;

FIG. 4 is a flowchart illustrating a method of monitoring ventilation adequacy, in accordance with an aspect of the present disclosure.

### DETAILED DESCRIPTION

Although the present disclosure will be described in terms of a specific embodiment, it will be readily apparent to those skilled in this art that various modifications, rearrangements and substitutions may be made without departing from the spirit of the present disclosure. The scope of the present disclosure is defined by the claims appended hereto.

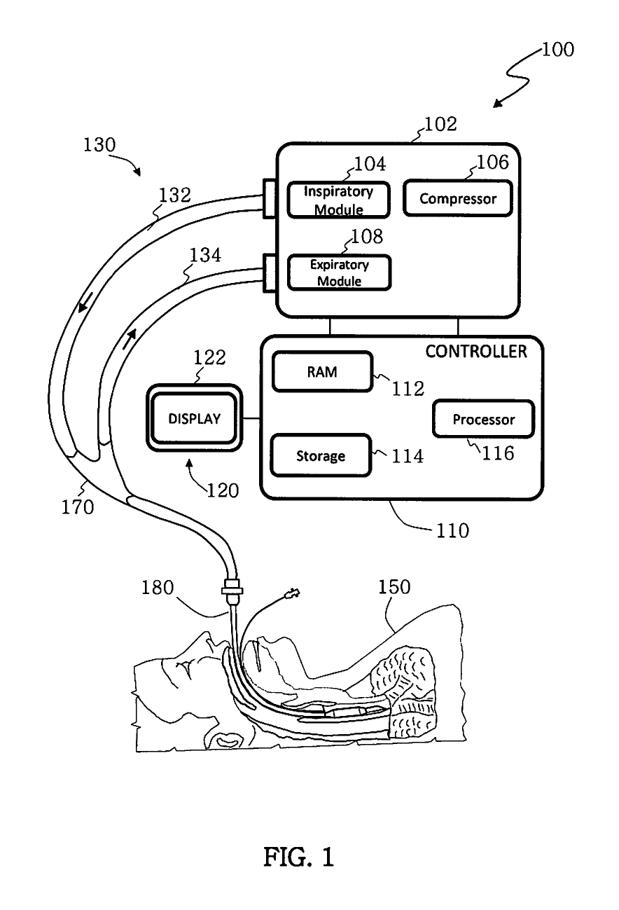

FIG. 1 is a diagram illustrating a ventilator 100 connected to a patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, breath types, view monitored parameters, etc.). Controller 110 may include memory 112, one or more processors 116, memory 114, and/or other components of the type commonly found in command and control computing devices. In the depicted example, operator interface 120 includes a display 122 that may be touch-sensitive and/or voice-activated, enabling the display 122 to serve both as an input and output device. Alternatively, a keyboard (not shown) or other data input device may be employed.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network via wired or wireless means.

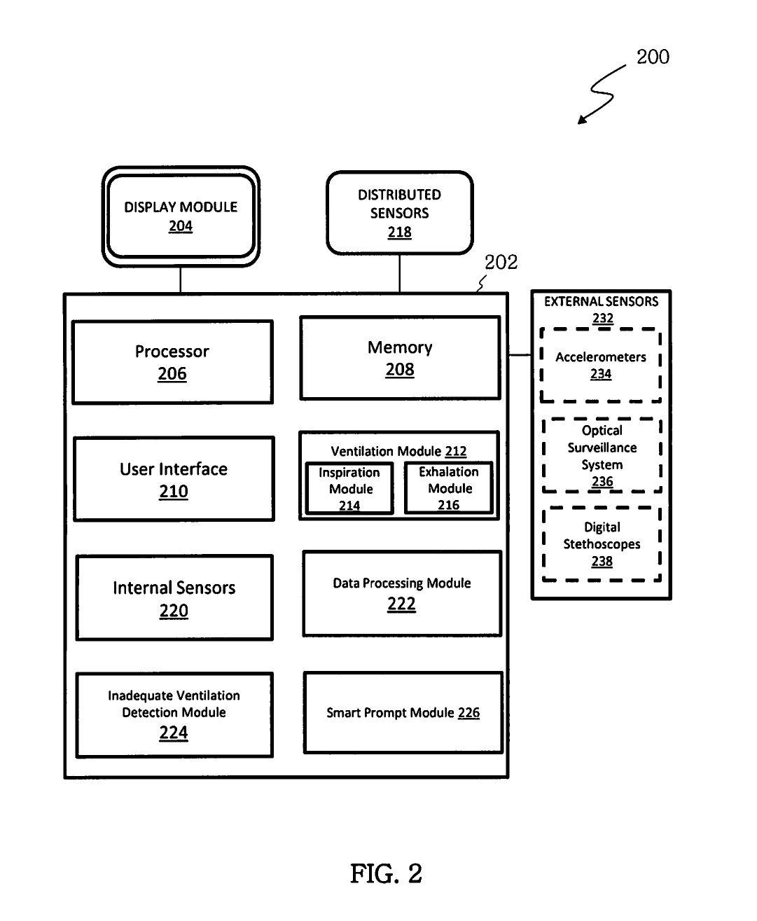

FIG. 2 is a block-diagram illustrating an embodiment of a ventilatory system 200 for monitoring and evaluating ventilatory parameters associated with an inadequate ventilation or asymmetric ventilation.

Ventilatory system 200 includes ventilator 202 with its various modules and components. That is, ventilator 202 may further include, for example, memory 208, one or more processors 206, user interface 210, and ventilation module 212 (which may further include an inspiration module 214 and an exhalation module 216). Memory 208 is defined as described above for memory 112. Similarly, the one or more processors 206 are defined as described above for one or more processors 116. Processors 206 may further be configured with a clock whereby elapsed time may be monitored by the ventilatory system 200. The ventilatory system 200 may also include a display module 204 communicatively coupled to ventilator 202. Display module 204 provides various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., including ventilatory data, alerts, patient information, parameter settings, etc.). The elements may include controls, graphics, charts, tool bars, input fields, smart prompts, etc. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed to the clinician in the form of graphs, wave representations, pie graphs, or other suitable forms of graphic display. For example, one or more smart prompts may be displayed on the GUI and/or display module 204 upon detection of an asymmetric chest wall movement or tidal volume displacement by the ventilator. Additionally or alternatively, one or more smart prompts may be communicated to a remote monitoring system coupled via any suitable means to the ventilatory system 200.

As used herein, the term “clinician” refers to any medical professional (i.e., doctor, surgeon, nurse, or the like) performing and/or monitoring and/or supervising a medical procedure involving the use of the embodiments described herein.

Ventilation module 212 may oversee ventilation of a patient according to prescribed ventilatory settings. For example, ventilation of a patient may be performed according to a series of ventilation parameters and/or ventilatory data based on a series of controlling equations. By way of general overview, the basic elements impacting ventilation may be described by the Equation of Motion as described in co-pending application Ser. No. 13/035,974, the entirety of which is incorporated herein by reference. Likewise the measurement and calculation of various parameters including, for example, pressure, flow and volume, respiratory compliance, respiratory resistance, pulmonary time constant, and normal resistance and compliance, are also described in co-pending application Ser. No. 13/035,974.

The ventilatory system 200 may also include one or more distributed sensors 218 communicatively coupled to ventilator 202. Distributed sensors 218 may communicate with various components of ventilator 202, e.g., ventilation module 212, internal sensors 220, data processing module 222, an inadequate ventilation detection module 224, and any other suitable components and/or modules. Distributed sensors 218 may detect changes in ventilatory parameters indicative of inadequate ventilation, for example. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors 218 may further include pressure transducers that may detect changes in circuit pressure (e.g., electromechanical transducers including piezoelectric, variable capacitance, or strain gauge). Alternatively or additionally, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Any distributed sensory device useful for monitoring changes in measurable parameters during ventilatory treatment may be employed in accordance with embodiments described herein.

Ventilator 202 may further include one or more internal sensors 220. Internal sensors 220 may communicate with various components of ventilator 202, e.g., ventilation module 212, internal sensors 220, data processing module 222, an inadequate ventilation detection module 224, and any other suitable components and/or modules. Internal sensors 220 may employ any suitable sensory or derivative technique for monitoring one or more parameters associated with the ventilation of a patient. The one or more internal sensors 220 may be placed in any suitable internal location, such as, for example, within the ventilatory circuitry or within components or modules of ventilator 202. For example, sensors may be coupled to the inspiratory and/or expiratory modules for detecting changes in, for example, circuit pressure and/or flow. Additionally or alternatively, internal sensors 220 may utilize optical or ultrasound techniques for measuring changes in ventilatory parameters. For example, a patient's expired gases may be monitored by internal sensors 220 to detect physiological changes indicative of the patient's condition and/or treatment. Internal sensors 220 may employ any suitable mechanism for monitoring parameters of interest in accordance with embodiments described herein.

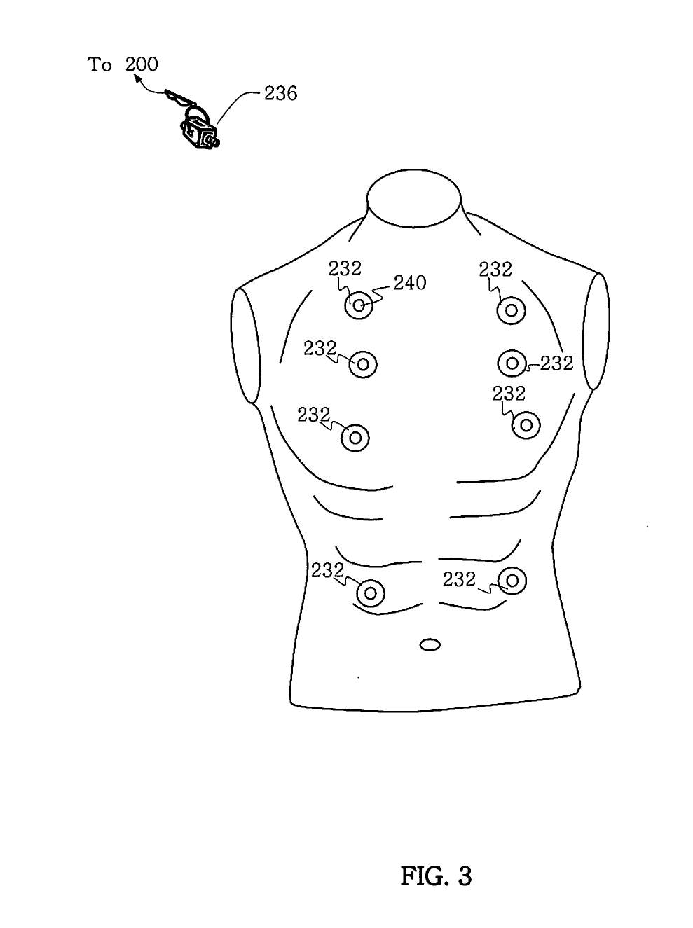

Referring now to FIGS. 2 and 3, ventilatory system 200 may further include one or more external sensors 232 that are configured to monitor localized chest wall movement (e.g., range of motion) of a patient's chest and lungs. For example, external sensors 232 may be accelerometers, image or video surveillance devices, audio sensors, motion capture or motion detection devices, electrical impedance sensors, ultrasound sensors, or other similar devices that are configured to monitor the magnitude and direction of chest wall movement. One or more indicators of the characteristics of displacement of a patient's lungs in conjunction with local tidal volume may be calculated based on the detected localized chest wall movement. For example, the direction and/or normalized amplitude of localized chest wall movement of each of the left and right lungs during a ventilatory cycle may be monitored and compared to determine a relative percentage of movement for each lung, e.g., 45% of the movement for the left lung and 55% of the movement for the right lung as typically found in a normal and healthy adult patient. In addition, for example, the direction and/or normalized amplitude of localized chest wall movement of the patient's chest and/or abdomen may be monitored and compared. The magnitude and/or relative amount of localized chest wall movement for each lung and/or the patient's abdomen may be correlated with a tidal volumetric displacement provided by the ventilator to determine the respective localized tidal volume displacement of the chest wall for each lung. For example, the percentage of movement may be correlated or equal to the percentage of the total tidal volume displacement of each of the right and left lungs, e.g., 45% movement for the left lung equals 45% of the total tidal volume displacement and 55% movement for the right lung equals 55% of the total tidal volume displacement. Alternatively, a calculation of the localized tidal volume displacement of each lung may be based on a combination of the localized chest wall movement with other respiratory parameters. External sensors 232 may also monitor the localized chest wall movement with respect to each individual lung lobe where the amount of localized chest wall movement of each lung lobe may be monitored and compared to determine the relative percentage of movement and/or local tidal volume displacement for each lung lobe.

External sensors 232 may communicate with various components of ventilator 202, e.g., ventilation module 212, internal sensors 220, data processing module 222, an inadequate ventilation detection module 224, and any other suitable components and/or modules. For example, external sensors 232 may monitor and detect changes in the localized chest wall movement of the lungs or lung lobes of the patient and send the detected sensory information to the ventilator 202 for storage in memory 208 and for processing by data processing module 222 and/or the inadequate ventilation detection module 224. Ventilator 202 may make the sensory information available to, for example, the clinician, a hand held device, a nursing station, a hospital server, a central monitoring station or another location set by a clinician via display module 204, user interface 210, smart prompt module 226 or another suitable notification method.

In some embodiments, an external device (not shown) such as a server or central processing device (not shown) may receive ventilatory data and/or parameters from ventilator 202, distributed sensors 218, internal sensors 220, external sensors 232 and/or other external devices, perform necessary processing on the received data, and transmit the results of the processing to ventilator 202 for use by ventilator 202. The server or central processing device may, for example, determine whether a threshold criteria is met and/or whether the received data is trending over time in a direction indicative of inadequate ventilation.

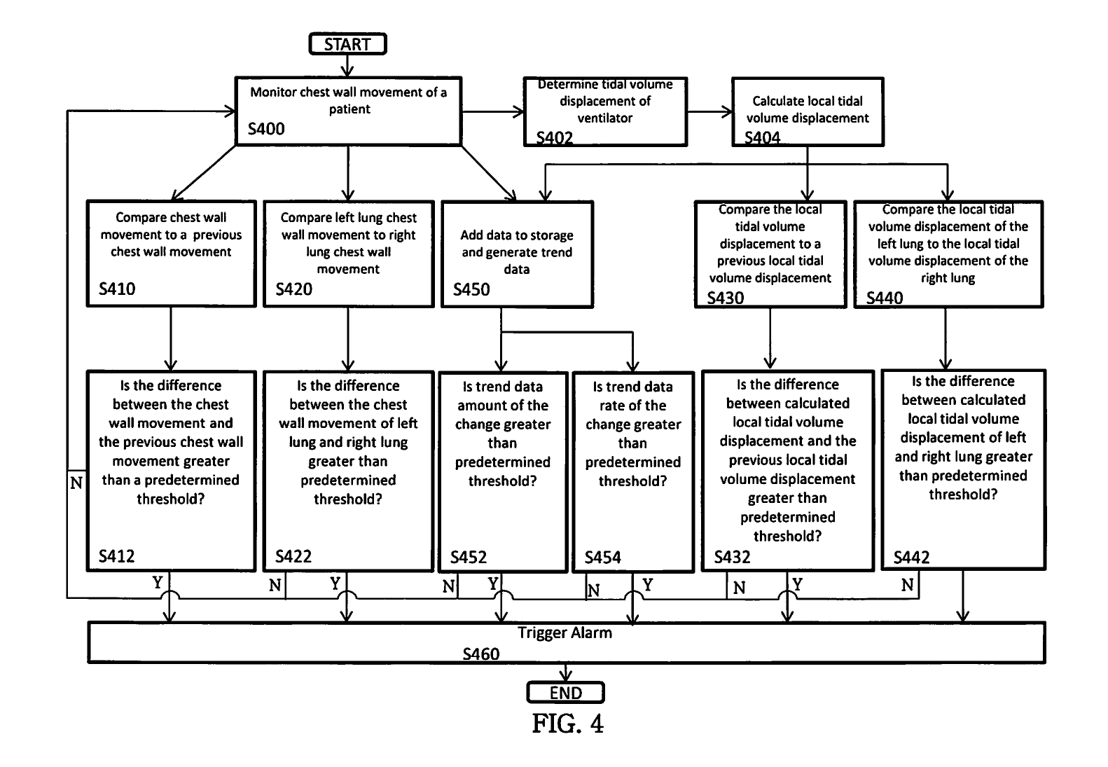

As an example, referring now to FIG. 4, the localized chest wall movement of a patient including both the chest and the abdomen may be monitored by the external sensors 232 to determine if a lung or lung lobe has collapsed or if a lung or lung lobe is experiencing a reduced or increased amount of movement and/or local tidal volume displacement. In step S400, external sensors 232 monitor the chest wall movement of each lung, lung lobe, and/or the patient's abdomen and in step S450 send the chest wall movement data to ventilator 202 for processing. In step S402, the tidal volume displacement into and out of the lungs is determined by the ventilator 202. In step S404, ventilator 202 calculates the local tidal volume displacement of each lung or lung lobe as described above for each ventilatory cycle. In step S410 the monitored chest wall movement is characterized and evaluated as well as compared to a previous chest wall movement and in step S430 the calculated local tidal volume displacement is evaluated and/or compared to a previous local tidal displacement. If the monitored chest wall movement and/or calculated local tidal volume displacement of one or both of the lungs, lung lobes, and/or the patient's abdomen does not meet a specified criterion and/or changes by a pre-determined threshold amount relative to a previously monitored or calculated amount (Steps S412, S423 respectively), for example, a change of 10%, 20%, 30%, 40%, 50%, another amount set by a clinician, or a threshold change derived from patient information, the ventilator 202 may trigger an alarm (Step S460) and provide to a clinician relevant data and/or recommendations, e.g., by smart prompt module 226, relating to the change. For example, the threshold criteria may be derived from patient information including one or more of a weighted combination of demographic, etiology, disease status, etc.

For example, external sensors 232 are particularly suited to the detection of asymmetrical ventilation of the lungs or of small changes in the local tidal displacement of each lung over one or more ventilatory cycles. For example, it may be determined that the two lungs and/or the lungs and the abdomen are moving in a paradoxical non-symmetrical and/or out of phase fashion. The alarm to the clinician may indicate that one or both of the lungs, lung lobes and/or the abdomen are experiencing abnormal localized chest movement or local tidal volume displacement indicative of, for example, a leak, a collapsed lung, etc. A clinician may set the alarm threshold amount for changes in localized chest movement and the local tidal volume displacement for each type of indication or the threshold change amount may be pre-set in the ventilatory system 200. For example, a threshold localized chest movement or local tidal volume displacement change of about 10%, 20%, 30%, 40%, 50%, or another amount of change set by a clinician, or a combination of indicators exceeding corresponding threshold criteria may be set as an alarm threshold and ventilator 202 may provide information or recommendations to the clinician indicative of a problem including, for example, a leak, a partial lung collapse, a stiffening of the lungs, a change in compliance, etc., while a threshold localized chest movement or local tidal volume displacement change greater than, for example, about 50% may be set as an alarm threshold and ventilator 202 may provide information or recommendations to the clinician indicative of a full lung collapse. Other threshold values may be used or set without departing from the scope of the present disclosure. The ventilator 202 may provide the clinician with different types of alarms (visual, audio, etc.) based on the particular alarm threshold that has been triggered. Furthermore, additional diagnostic tools (such as Electromagnetic Navigation Bronchoscopy) may be utilized to investigate or correlate the high level macro findings with more specific micro level information and/or data.

In one example, referring now to steps S420 and S422, if the ventilation tubing system 130 were to migrate from a position where a distal end of patient interface 180 is located above the main carina in the bronchial tree where both the left and right lungs are being ventilated by the ventilator 202, to a position where the distal end of patient interface 180 is located below the main carina in one of the lungs where only one lung is being ventilated by the ventilator 202, external sensors 232 positioned on the chest of the patient sense a reduced localized chest movement of the lung not being ventilated and an increased localized chest movement of the lung that is ventilated. In this case, if the difference between the localized chest movement of one lung relative to the other meets or exceeds a pre-determined threshold (Step S422), e.g., 30%, 40% or another threshold value set by the clinician, ventilator 202 triggers an alarm (Step S460). Alternatively or additionally, with reference to Steps S440 and S442 if the difference between a calculated local tidal volume displacement of each lung based on the localized chest movement of each lung meets or exceeds a pre-determined threshold, ventilator 202 triggers an alarm. The alarm could also provide to the clinician an indication or recommendation that it is likely the patient interface 180 of tubing system 130 has migrated down into one of the lungs.

As should be appreciated, with reference to the Equation of Motion, ventilatory parameters are highly interrelated and, according to embodiments, may be either directly or indirectly monitored. That is, parameters may be directly monitored by one or more sensors, as described above, or may be indirectly monitored by derivation according to the Equation of Motion.

In some embodiments, changes in the monitored localized chest movement or calculated local tidal volume displacement of the lungs may be correlated with other ventilatory parameters to determine whether a general declining trend is occurring. For example, when one or more ventilatory parameters, in addition to the monitored localized chest movement or calculated local tidal volume displacement are declining, but no parameter has individually declined enough to trigger an alarm threshold, the ventilator 202 may still trigger an alarm to the clinician. Such an alarm could provide a clinician with an early indication that an abnormal event is occurring with the patient and may allow the clinician to provide early treatment to the patient prior to any individual parameter declining past an alarm threshold level.

Algorithms for determination of respiratory mechanics (e.g., using a Kalman filter or least square methods) may be utilized to estimate and monitor a patient's respiratory resistance and compliance, and other patient respiratory parameters such as indicators of respiratory effort, spirometry, peak inspiratory pressure, peak expiratory flow, breath rate, etc., may be collected for a combinatorial and correlative analysis. Each parameter may be stored in memory 208 for later use by ventilator 202 (Step S450). Data processing module 222 and inadequate ventilation detection module 224 analyze the data stored in memory 208 and correlate the stored data with the monitored localized chest movement and calculated local tidal volume displacement of the lungs to detect correlative patterns between changes in respiratory (e.g., resistance and/or compliance) and localized chest wall dynamics or local tidal volume changes and determine potential asymmetrical ventilation.

In some embodiments, inadequacy of ventilation treatment may be measured by trending data (Step S450) gathered by the ventilator 202 and stored in memory 208. For example, monitored localized chest movement data and/or calculated local tidal volume displacement data may be stored in memory 208 and trended by ventilator 202 in a windowed correlation, e.g., a correlation calculated over a defined time frame, between previously monitored localized chest movement and/or calculated local tidal volume displacement data to determine if a general downward trend has occurred. If a threshold amount of change (Step S452) over a defined time frame has occurred, an alarm may be triggered by the ventilator to indicate to a clinician that a general decline in lung function over time has occurred. Alternatively, or additionally, if a threshold rate of change (Step S454) over a defined time frame has occurred, an alarm may be triggered by the ventilator to indicate to a clinician that a rapid decline in the patient's lung function is occurring.

Trending may also be used to correlate other respiratory parameters, such as, for example, resistance and compliance, with the measured localized chest movement and/or calculated local tidal volume displacement to provide a diagnostic predictive indicator of a patient's abnormal or failing lung function. In one embodiment, for example, an increase in total inspiratory airway resistance coincidental with a decrease in chest wall displacement or magnitude on the right side of the patient may indicate the presence of a mucous plug or other problem causing obstruction of gas flow to the right side. Absent an external sensor 232 on the patient's chest wall (or other measuring system) the determination of the location of an airway problem by a clinician may be particularly difficult. The external sensors 232 provide the clinician with an accurate and easily identifiable source of information relating to the movement of each side, or lung, of a patient's chest. In addition, external sensors 232 may detect when gasses are not being correctly exhausted from the patient's lungs during ventilation. For example, a mucous plug or other obstruction may cause gas from one lung to flow into the other lung during expiration. External sensors 232 may detect that one lung is compressing while the other lung is expanding and indicate or send an alarm to a clinician regarding the asymmetrical nature of the patient's lung movement.

In some embodiments, a correlation coefficient may be used as a robustness weighting multiplicative factor to be applied to quantitative measures of localized chest movement differences, to corroborate local tidal volume displacement deviations or differences of the lungs or lung lobes with concomitant trends in respiratory mechanics such as, for example, compliance or resistance, and/or paradoxical thoracic-abdominal movement indicative of respiratory fatigue.

In some embodiments, when a clinician is attempting to wean a patient off of the use of ventilator 202, external sensors 232 provide monitoring of the localized chest wall movement to ventilator 202 to allow ventilator 202 to determine a greater inference of when the patient is fatiguing. For example, external sensors 232 may sense abnormal localized chest wall movements during weaning that may be indicative of a chest collapse, of muscle fatigue (e.g. the diaphragm), of the wrong set of muscles acting during breathing, or other similar weaning problems. Upon detection of any of these situations, the ventilator 202 would infer that there is a problem with the weaning process based on the sensed values and trigger an alarm to the clinician with this information. The ventilator 202 may also automatically initiate one or more ventilatory cycles, i.e., inspiration and expiration cycles, to ensure that the patient receives proper ventilation until the clinician arrives.

In some embodiments, external sensors 232 may alternatively be positioned within the patient's lungs or may be surgically inserted into the patient's chest.

In some embodiments, external sensors 232 are accelerometers 234 located on an external surface of the patient's chest. For example, accelerometers 234 may be located on the left or right side of the chest, on an upper or lower portion of the chest, on the abdomen, or any other location on the patient that would provide an indication of chest movement. For example, an accelerometer 234 may be located adjacent each lung lobe to provide monitoring of both the lungs and the lung lobes to the ventilator 202. Any combination of the above positioning of accelerometers 234 may be used to provide an accurate mapped reading and monitoring of the localized chest wall movement of a patient. Accelerometers 234 are constructed with sufficient accuracy to detect small differences in localized chest wall movements during a ventilatory cycle. On a neonatal patient, accelerometers 234 having increased accuracy and resolution may be used to measure the smaller differences in chest movement associated with a new born baby. Accelerometers 234 positioned as described above are particularly suited to the detection of asymmetric ventilation. Positioning accelerometers 234 on the exterior chest wall of the patient provides the ventilator with sensory information for the monitoring of localized chest wall movement of each lung and for calculation of the relative local tidal volume displacement differences between the lungs and during successive ventilatory cycles. Output data from accelerometers 234 may be sent to ventilator 202 and stored in memory 208 for use during a corroborative analysis, as described above.

In some embodiments, ventilatory system 200 includes an optical surveillance system 236 that views a patient's chest during each ventilatory cycle. The optical surveillance system 236 utilizes pattern recognition or image processing techniques to monitor the localized chest wall movement of a patient. In one embodiment, external sensors 232 may include a dot, ball, or other mechanism 240 for providing optical surveillance system 236 with accurate tracking points to monitor the patient's localized chest movement. Output data from optical surveillance system 236 may be sent to ventilator 202 and stored in memory 208 for use during a corroborative analysis, as described above.

In some embodiments, the external sensors 232 may be audio sensors, such as digital stethoscopes 238. Digital stethoscopes 238 are configured to monitor the sound of local tidal volume displacement within the chest of the patient to determine the local tidal displacement during a ventilatory cycle. Digital stethoscopes 238 are particularly suited to the detection of wheezing or other abnormal sounds in the lungs during ventilation which may indicate an increase in airway resistance due to, for example, a partial blockage, narrowing of passageways in the bronchial tree due to inflammation, etc. By measuring differences in the sound of the chest between ventilation cycles and/or using this information in conjunction with other concomitant measurements collected in real-time or pseudo-real time, the local tidal volume displacement of each lung may be determined and any changes relative to a prior ventilation cycle may also be determined for use by the ventilator 202. Output data from digital stethoscopes 238 may be sent to ventilator 202 and stored in memory 208 for use during a corroborative analysis, as described above.

In some embodiments, external sensors 232 may be electrical impedance sensors (not shown) configured to determine localized chest wall movement by monitoring the changes in impedance of the chest wall and lungs of the patient during ventilation.

In some embodiments, external sensors 232 may be ultrasound sensors (not shown) configured to determine localized chest wall movement by monitoring changes in ultrasound images or data of the chest wall and lung movement during ventilation. For example, image processing may be performed on an ultrasound image or ultrasound data may be analyzed by ventilator 202.

The data/information, indicators, and/or metrics received by and analyzed by the ventilator 202 may be displayed in various forms to the clinician to assist in monitoring the patient. For example, ventilator 202 may include a smart prompt module 226 for generating a prompt or alarm. As may be appreciated, multiple ventilatory parameters may be monitored and evaluated in order to detect an occurrence of inadequate ventilation. In addition, when inadequate ventilation occurs, many clinicians may not be aware of adjustments to ventilatory parameters or to patient interface 180 that may reduce or eliminate the inadequate ventilation. Upon detection of inadequate ventilation, the smart prompt module 226 may be configured to notify the clinician that inadequate ventilation is occurring and/or to provide recommendations to the clinician for mitigating the inadequate ventilation. For example, smart prompt module 226 may be configured to notify the clinician by displaying a smart prompt on display monitor 204 and/or within a window of the GUI. According to additional embodiments, the smart prompt may be communicated to and/or displayed on a remote monitoring system communicatively coupled to ventilatory system 200. According to alternative embodiments, the smart prompt is any audio and/or visual notification. Alternatively, in an automated embodiment, the smart prompt module 226 may communicate with a ventilator control system so that the recommendation may be automatically implemented to mitigate the inadequate ventilation. A description of the function of a similar smart prompt module is described in further detail in co-pending application Ser. No. 13/035,974.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure.

The foregoing examples illustrate various aspects of the present disclosure and practice of the methods of the present disclosure. The examples are not intended to provide an exhaustive description of the many different embodiments of the present disclosure. Thus, although the foregoing present disclosure has been described in some detail by way of illustration and example for purposes of clarity and understanding, those of ordinary skill in the art will realize readily that many changes and modifications may be made thereto without departing form the spirit or scope of the present disclosure.

While several embodiments of the disclosure have been shown in the drawings and described in detail hereinabove, it is not intended that the disclosure be limited thereto, as it is intended that the disclosure be as broad in scope as the art will allow. Therefore, the above description and appended drawings should not be construed as limiting, but merely as exemplifications of particular embodiments. Those skilled in the art will envision other modifications within the scope and spirit of the claims appended hereto.

What is claimed is:

1. A ventilatory system comprising:
a ventilator; and
at least one sensor in communication with the ventilator and configured to monitor a chest wall or abdominal movement of a patient during ventilation,
wherein the ventilator is configured to provide an alarm to a clinician upon detection by the at least one sensor of a threshold change in the chest wall or abdominal movement of the patient.

2. The ventilatory system according to claim 1, wherein the at least one sensor is an accelerometer.

3. The ventilatory system according to claim 1, wherein the at least one sensor is located adjacent at least one lung lobe.

4. The ventilatory system according to claim 1, wherein the at least one sensor is an optical imaging system configured to capture images of the patient's chest wall or abdominal movement during ventilation.

5. The ventilatory system according to claim 1, wherein the at least one sensor is an audio sensor.

6. The ventilatory system according to claim 1, wherein the threshold change is a threshold change between the monitored chest wall movement and a previously monitored chest wall movement.

7. The ventilatory system according to claim 1, wherein the threshold change is a threshold change between the monitored chest wall movement and an external reference point.

8. A method of detecting inadequate ventilation of a patient comprising:
monitoring a chest wall movement of the patient;
determining if a threshold change in the chest wall or abdominal movement of the patient has occurred; and
triggering an alarm if the threshold change in the chest wall or abdominal movement of the patient has occurred.

9. The method according to claim 8, further comprising:
comparing the chest wall or abdominal movement of the patient to a previous chest wall or abdominal movement of the patient; and
triggering the alarm if the difference between the chest wall or abdominal movement and the previous chest wall movement exceeds a pre-determined threshold.

10. The method according to claim 8, further comprising:
determining a tidal volume displacement of a ventilator;
calculating a local tidal volume displacement based on the chest wall movement of the patient and the determined tidal volume displacement of the ventilator;
comparing the calculated tidal volume displacement to a previous tidal volume displacement of the patient; and
triggering an alarm if the difference between the calculated tidal volume displacement and the previous tidal volume displacement exceeds a pre-determined threshold.

11. The method according to claim 10, wherein monitoring the chest wall movement includes separately monitoring the chest wall movement of each lung, and wherein the local tidal volume displacement of each lung is calculated based on a comparison of an amount of the chest wall movement of each lung and the determined tidal volume displacement of the ventilator.

12. The method according to claim 8, wherein monitoring the chest wall movement of the patient includes separately monitoring the chest wall movement of each lung of the patient, the method further comprising:
comparing the chest wall movement of each lung; and
triggering an alarm if the difference between the chest wall movement of each lung exceeds a pre-determined threshold.

13. The method according to claim 8, wherein monitoring the chest wall movement of the patient includes separately monitoring the chest wall movement of at least one lung lobe of the patient, the method further comprising:
comparing the chest wall movement of the at least one lung lobe to a previous chest wall movement of the at least one lung lobe; and
triggering an alarm if the difference between the monitored chest wall movement of the at least one lung lobe and the previous chest wall movement of the at least one lung lobe exceeds a pre-determined threshold.

14. The method according to claim 8, further comprising:
storing monitored chest wall or abdominal movement data in a non-transitory storage medium;
generating chest wall movement or abdominal trend data based on the stored chest wall movement data; and
triggering an alarm if at least one of a rate of change and an amount of change of the chest wall or abdominal movement trend data exceeds a pre-determined threshold.

15. The method according to claim 14, further comprising:
storing respiratory parameter data in the non-transitory storage medium;
correlating chest wall movement trend data with the respiratory parameter data; and
triggering an alarm if the correlation between the respiratory parameter data and the chest wall movement trend data meets a predetermined threshold.

16. The method according to claim 10, further comprising:
storing the calculated local tidal volume displacement data in a non-transitory storage medium;
generating local tidal volume displacement trend data based on the stored local tidal volume displacement data; and
triggering an alarm if at least one of a rate of change and an amount magnitude of change of the local tidal volume displacement trend data exceeds a pre-determined threshold.

17. The method according to claim 16, further comprising:
storing respiratory parameter data in the non-transitory storage medium;
correlating local tidal volume displacement trend data with the respiratory parameter data; and
triggering an alarm if the correlation between the respiratory parameter data and the local tidal volume displacement trend data meets a predetermined threshold.

18. A non-transitory computer-readable storage medium encoded with a program that, when executed by a processor detects inadequate ventilation of a patient and causes the processor to perform the steps of:
monitoring a chest wall or abdominal movement of the patient;
determining if a threshold change in the chest wall or abdominal movement of the patient has occurred; and
triggering an alarm if the threshold change in the chest wall or abdominal movement of the patient has occurred.

19. The non-transitory computer-readable storage medium of claim 18, further performing steps of:
comparing the chest wall or abdominal movement of the patient to a previous chest wall movement of the patient; and
triggering the alarm if the difference between the chest wall or abdominal movement and the previous chest wall movement exceeds a pre-determined threshold.

20. The non-transitory computer readable storage medium of claim 18, wherein monitoring the chest wall movement of the patient includes separately monitoring the chest wall movement of each lung of the patient, and performing steps of:
comparing the chest wall movement of each lung; and
triggering an alarm if the difference between the chest wall movement of each lung exceeds a pre-determined threshold.
